(19) United States
(12) Patent Application Publication
(10) Pub. No.: US 2025/0002900 A1
(43) Pub. Date: Jan. 2, 2025

(54) Methods for Producing DNA Libraries and Uses Thereof

(71) Applicant: QIAGEN SCIENCES, LLC, Germantown, MD (US)

(72) Inventors: Eric LADER, Bloomfield Hills, MI (US); Zhong WU, Poolesville, MD (US); Yexun WANG, San Diego, CA (US); Quan PENG, Clarksburg, MD (US)

(21) Appl. No.: 18/700,951

(22) PCT Filed: Oct. 15, 2021
(86) PCT No.: PCT/US2021/055174

§ 371 Date: Apr. 12, 2024

(51) Int. Cl. C12N 15/10

(52) U.S. Cl. CPC C12N 15/1065; C12N 15/1068

(57) ABSTRACT

Disclosed herein are methods for producing DNA libraries by incorporating dUTPs into DNA fragments and treating with uracil-DNA glycosylase and kits for preparing the DNA libraries. The DNA libraries are advantageous for next generation sequencing.

### REFERENCE TO SEQUENCE LISTING SUBMITTED ELECTRONICALLY

The content of the electronically submitted sequence listing in ASCII text file (Name: 2495-0017WO01_Sequence_Listing_ST25.txt; Size: 2 KB; and Date of Creation: Oct. 14, 2021) filed with the application is incorporated herein by reference in its entirety.

### FIELD

Disclosed herein are methods for producing DNA libraries by incorporating dUTPs into DNA fragments and treating with uracil-DNA glycosylase and kits for preparing the DNA libraries. The DNA libraries are advantageous for next generation sequencing.

### BACKGROUND OF THE INVENTION

Efficient and quality DNA library construction is important for next generation sequencing. Improved methods for generating DNA libraries are needed, with high numbers of original molecules recovery, primer multiplex level, and coverage uniformity.

### BRIEF SUMMARY OF THE INVENTION

Disclosed herein are methods of preparing a DNA library, comprising (a) ligating an adapter to a target DNA molecule to generate a ligation product, wherein the adapter comprises one or more deoxyuridines and a unique molecular index (UMI); (b) treating the ligation product with a uracil-DNA glycosylase (UDG); and (c) amplifying the target DNA molecule attached to the adapter in the ligation product of (b) by single primer extension with a target specific primer to generate a target amplification product, wherein the target specific primer comprises one or more deoxyuridines and a target specific sequence.

Also disclosed herein are methods of preparing a DNA library, comprising amplifying a target DNA molecule with a single target specific primer to generate a target amplification product, wherein the target specific primer comprises a tag sequence, one or more deoxyuridines, and a target specific sequence. In some embodiments, the adapter is a single adapter that is annealed to the target DNA molecule and extended, wherein the adapter comprises a UMI and a target specific sequence that does not contain a deoxyuridine. In some embodiments, a pair of adapters are annealed to the target DNA molecule and extended, wherein each adapter of the pair of adapters comprises a UMI, a first or second target specific sequence, and one or more deoxyuridines. In some embodiments, an adapter has been ligated to the target DNA molecule to generate a ligation product, wherein the adapter comprises one or more deoxyuridines and a unique molecular index (UMI). Also disclosed herein are methods of preparing a DNA library, comprising (a) extending a target DNA molecule attached to an adapter comprising one or more deoxyuridines and a unique molecular index (UMI); (b) treating the extension product of (a) with a uracil-DNA glycosylase (UDG); and (c) amplifying the extension product of (b) with a target specific primer to generate a target amplification product, wherein the target specific primer comprises one or more deoxyuridines and a target specific sequence.

In some embodiments, the target specific sequence comprises one or more deoxyuridines.

In some embodiments, the target DNA molecule has been end-repaired. In some embodiments, the target DNA molecule has been adenylated.

In some embodiments, the target specific primer comprises a tag sequence comprising at least 2 deoxyuridines and a target specific sequence comprising 0-4 deoxyuridines in a 5′ to 3′ direction.

In some embodiments, the methods further comprise treating the target amplification product with a UDG and then amplifying the target amplification product with a universal primer to generate a second target amplification product, wherein the universal primer does not comprise a deoxyuridine. In some embodiments, the universal primer comprises a sample index.

In some embodiments, the ligating is performed at a pH of 8-9. In some embodiments, the ligating is performed at a PEG concentration of 4% PEG to less than 10% PEG.

In some embodiments, the amplifying is performed with an antibody based hotstart thermostable polymerase.

In some embodiments, the methods further comprise purifying the second amplification product with beads. In some embodiments, the beads are solid phase reversible immobilization (SPRI) beads.

The DNA libraries can be used for next generation sequencing, profiling of DNA variants, human identity or paternity testing, pain or ADME pharmacogenomics, or detection of a genetic disease.

Disclosed herein are DNA libraries made by the methods disclosed herein.

Disclosed herein are kits comprising one or more of the components used in the methods disclosed herein. Thus, disclosed herein are kits comprising a uracil-DNA glycosylase (UDG), an adapter comprising one or more deoxyuridines and a unique molecular index (UMI), a universal primer, and an antibody based hotstart polymerase. In some embodiments, the kits can further comprise a ligase and a ligation buffer capable of providing a pH of 8 to 9 to a ligation reaction. In some embodiments, the ligation buffer is capable of providing a PEG concentration of 4% to less than 10% to the ligation reaction. In some embodiments, the kits further comprise solid phase reversible immobilization (SPRI) beads. In some embodiments, the kits further comprise a target specific primer comprising one or more deoxyuridines and a target specific sequence.

### BRIEF DESCRIPTION OF THE DRAWINGS/FIGURES

FIG. 1. Exemplary workflow: not to scale. “A” denotes an A base,

FIG. 2. Another exemplary workflow: not to scale. “A” denotes an A base; “U” denotes a U base; and “X” denotes where the U base is removed by UDG (an abasic site).

FIG. 3. Another exemplary workflow: not to scale. “A” denotes an A base; “U” denotes a U base; and “X” denotes where the U base is removed by UDG (an abasic site).

FIG. 4. Ligation buffer of pH 8.5 with 6% PEG in the ligation reaction had similar or slightly lower UMI compared to ligation buffer of pH 7.6 with 13.2% PEG, suggesting high pH ligation buffer of low PEG in the reaction could achieve similar ligation efficiency as regular pH ligation buffer with higher PEG in the reaction.

FIG. 5. The ligation buffer with high pH had much more UMI compared to the buffer with low pH suggesting the high pH ligation buffer can be used in targeted DNA panel workflow.

FIG. 6. Universal PCR was carried out either with chem hotstar Taq for 2 min at 62° C. anneal/extension in each cycle or with Ab hotstart Taq for 1 min or 30 sec at 62° C. anneal/extension. General panel performance of specificity and uniformity looked similar among these conditions.

FIGS. 7A-7B. Sample Bioanalyzer images of QIAseq Targeted DNA Pro libraries for Illumina instruments. The size of the majority of the library fragments are between 200-1000 bp. FIG. 7A. Library without over-amplification. FIG. 7B. Library with over-amplification as indicated by the “larger fragment” peak.

### DETAILED DESCRIPTION OF THE INVENTION

Single Primer Extension (SPE) NGS library construction is a very successful technology commercially but can be improved for high throughput, fast turnaround laboratories. Single primer extension (SPE), like most other methods, relies on cleanup with magnetic SPRI beads for adapter and primer removal, which is a low throughput, variable, time consuming step and can result in sample loss due to the use of SPRI beads prior to target amplification.

Disclosed herein are methods of using dU substituted oligos and UNG digestion to replace all but one final bead cleanup. This makes automation much easier, recovers far more original molecules (no loss in bead steps), and saves hours of processing time.

Ligation reactions also pose problems to automation. Generally, ligation requires high concentrations of PEG, causing high viscosity and problems in pipetting and mixing. Disclosed herein are methods that substitute with a high pH ligation buffer that allows the removal of most of the PEG such that final ligation reactions contain less than 10% PEG, aiding in efficient automation and enhanced reproducibility.

Finally, disclosed herein are methods in which throughput of library construction is vastly accelerated by replacing a chemical hotstart taq polymerase with an antibody-based fast-cycling alternative. Finding an amplification mix and cycling conditions that are compatible with the NGS demands of high numbers of primers, high uniformity, and an enzyme tolerant of dU substitution in primers required significant amount of work and analyses.

Accordingly, disclosed herein are methods of preparing a DNA library, comprising (a) ligating an adapter to a target DNA molecule to generate a ligation product, wherein the adapter comprises one or more deoxyuridines and a unique molecular index (UMI); (b) treating the ligation product with a uracil-DNA glycosylase (UDG); and (c) amplifying the target DNA molecule attached to the adapter in the ligation product of (b) by single primer extension with a target specific primer to generate a target amplification product, wherein the target specific primer comprises one or more deoxyuridines and a target specific sequence. In some embodiments, the ligated adapter in (a) is single-stranded and becomes double-stranded by extension, thus copying the UMI to both strands of the target DNA molecule.

Also disclosed herein are methods of preparing a DNA library, comprising amplifying a target DNA molecule with a single target specific primer to generate a target amplification product, wherein the target specific primer comprises a tag sequence, one or more deoxyuridines, and a target specific sequence. In some embodiments, the adapter is a single adapter that is annealed to the target DNA molecule and extended, wherein the adapter comprises a UMI and a target specific sequence that does not contain a deoxyuridine. In some embodiments, a pair of adapters are annealed to the target DNA molecule and extended, wherein each adapter of the pair of adapters comprises a UMI, a first or second target specific sequence, and one or more deoxyuridines. In some embodiments, an adapter has been ligated to the target DNA molecule to generate a ligation product, wherein the adapter comprises one or more deoxyuridines and a unique molecular index (UMI). Also disclosed herein are methods of preparing a DNA library, comprising (a) extending a target DNA molecule attached to an adapter comprising one or more deoxyuridines and a unique molecular index (UMI); (b) treating the extension product of (a) with a uracil-DNA glycosylase (UDG); and (c) amplifying the extension product of (b) with a target specific primer to generate a target amplification product, wherein the target specific primer comprises one or more deoxyuridines and a target specific sequence. In some embodiments, the target specific sequence comprises one or more deoxyuridines.

In some embodiments, the target DNA molecule has been end-repaired. In some embodiments, the target DNA molecule has been adenylated.

In some embodiments, the target specific primer comprises a tag sequence comprising at least 2 deoxyuridines and a target specific sequence comprising 0-4 deoxyuridines in a 5′ to 3′ direction.

In some embodiments, the methods further comprise treating the target amplification product with a UDG and then amplifying the target amplification product with a universal primer to generate a second target amplification product, wherein the universal primer does not comprise a deoxyuridine. In some embodiments, the universal primer comprises a sample index.

In some embodiments, the ligating is performed at a pH of 8-9. In some embodiments, the ligating is performed at a PEG concentration of 4% PEG to less than 10% PEG.

In some embodiments, the amplifying is performed with an antibody based hotstart polymerase.

In some embodiments, the methods further comprise purifying the second amplification product with beads. In some embodiments, the beads are solid phase reversible immobilization (SPRI) beads.

The DNA libraries can be used for next generation sequencing, profiling of DNA variants, human identity or paternity testing, pain or ADME pharmacogenomics, or detection of a genetic disease.

Disclosed herein are DNA libraries made by the methods disclosed herein.

Disclosed herein are kits comprising a uracil-DNA glycosylase (UDG), an adapter comprising one or more deoxyuridines and a unique molecular index (UMI), a universal primer, and an antibody based thermostable polymerase. In some embodiments, the kits can further comprise a ligase and a ligation buffer capable of providing a pH of 8 to 9 to a ligation reaction. In some embodiments, the ligation buffer is capable of providing a PEG concentration of 4% to less than 10% to the ligation reaction. In some embodiments, the kits further comprise solid phase reversible immobilization (SPRI) beads. In some embodiments, the kits further comprise a target specific primer comprising one or more deoxyuridines and a target specific sequence.

The term “sample” can include RNA, DNA, a single cell, multiple cells, fragments of cells, or an aliquot of body fluid, taken from a subject (e.g., a mammalian subject, an animal subject, a human subject, or a non-human animal subject). Samples can be selected by one of skill in the art using any known means known including but not limited to centrifugation, venipuncture, blood draw, excretion, swabbing, biopsy, needle aspirate, lavage sample, scraping, surgical incision, laser capture microdissection, gradient separation, or intervention or other means known in the art. The term “mammal” or “mammalian” as used herein includes both humans and non-humans and include but is not limited to humans, non-human primates, canines, felines, murines, bovines, equines, and porcines.

As used herein, the term “biological sample” is intended to include, but is not limited to, tissues, cells, biological fluids and isolates thereof, isolated from a subject, as well as tissues, cells, and fluids present within a subject.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

A single cell suspension can be obtained using standard methods known in the art including, for example, enzymatically using trypsin or papain to digest proteins connecting cells in tissue samples or releasing adherent cells in culture, or mechanically separating cells in a sample. Samples can also be selected by one of skill in the art using one or more markers known to be associated with a sample of interest.

Methods for manipulating single cells are known in the art and include fluorescence activated cell sorting (FACS), micromanipulation and the use of semi-automated cell pickers (e.g., the Quixell™ cell transfer system from Stoelting Co.). Individual cells can, for example, be individually selected based on features detectable by microscopic observation, such as location, morphology, or reporter gene expression.

Once a desired sample has been identified, the sample is prepared and the cell(s) are lysed to release cellular contents including DNA and RNA, such as gDNA and mRNA, using methods known to those of skill in the art. Lysis can be achieved by, for example, heating the cells, or by the use of detergents or other chemical methods, or by a combination of these. Any suitable lysis method known in the art can be used. Nucleic acids from a cell such as DNA or RNA can be isolated using methods known to those of skill in the art.

The term “target DNA molecule” or “target nucleic acid molecule” refers to a DNA or nucleic acid molecule of interest, e.g., a DNA fragment, to be analyzed. In some embodiments, the target DNA molecule comprises a target sequence (e.g., a known or predetermined sequence) and an adjacent sequence to be determined. The target DNA molecules can be obtained from a sample(s) or a single cell(s).

The term “DNA” refers to chromosomal DNA, plasmid DNA, phage DNA, viral DNA that is single stranded (ssDNA) or double stranded (dsDNA), or cDNA. DNA can be obtained from prokaryotes or eukaryotes.

The term “genomic DNA” or gDNA” refers to chromosomal DNA.

The term “DNA fragment(s)” refer to DNA that are fragmented, naturally or by, but not limited to, enzymes or sonication.

The term “messenger RNA” or “mRNA” refers to an RNA that is without introns and that can be translated into a polypeptide.

The term “cDNA” refers to a DNA that is complementary or identical to an mRNA, in either single stranded or double stranded form. Methods for obtaining cDNA are well known in the art.

The term “polynucleotide(s)” or “oligonucleotide(s)” refers to nucleic acids such as DNA molecules and RNA molecules and analogs thereof (e.g., DNA or RNA generated using nucleotide analogs or using nucleic acid chemistry). As desired, the polynucleotides can be made synthetically, e.g., using art-recognized nucleic acid chemistry or enzymatically using, e.g., a polymerase, and, if desired, can be modified. Typical modifications include methylation, biotinylation, and other art-known modifications. In addition, a polynucleotide can be single-stranded or double-stranded and, where desired, linked to a detectable moiety. In some aspects, a polynucleotide can include hybrid molecules, e.g., comprising DNA and RNA.

“G,” “C,” “A,” “T” and “U” each generally stands for a nucleotide that contains guanine, cytosine, adenine, thymidine and uracil as a base, respectively. However, it will be understood that the term “ribonucleotide” or “nucleotide” can also refer to a modified nucleotide or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in nucleotide sequences by a nucleotide containing, for example, inosine. In another example, adenine and cytosine anywhere in the oligonucleotide can be replaced with guanine and uracil, respectively, to form G-U Wobble base pairing with the target mRNA. Sequences containing such replacement moieties are suitable for the compositions and methods described herein. In some embodiments, as disclosed herein, the dUTP nucleotide can be incorporated into DNA as dUMP, also referred to as dU or U.

As used herein, the terms “ligating,” “ligation,” and their derivatives refer generally to the act or process for covalently linking two or more molecules together, for example, covalently linking two or more nucleic acid molecules to each other, and generating a ligation product. In some embodiments, ligation includes joining nicks between adjacent nucleotides of nucleic acids. In some embodiments, ligation includes forming a covalent bond between an end of a first and an end of a second nucleic acid molecule. In some embodiments, for example embodiments wherein the nucleic acid molecules to be ligated include conventional nucleotide residues, the litigation can include forming a covalent bond between a 5′ phosphate group of one nucleic acid and a 3′ hydroxyl group of a second nucleic acid thereby forming a ligated nucleic acid molecule. In some embodiments, any means for joining nicks or bonding a 5′phosphate to a 3′ hydroxyl between adjacent nucleotides can be employed. In an exemplary embodiment, an enzyme such as a ligase can be used. In some embodiments, a target DNA molecule can be ligated to an adapter to generate a ligation product comprising an adapter ligated to a target DNA molecule.

As used herein, “ligase” and its derivatives, refers generally to any agent capable of catalyzing the ligation of two substrate molecules. In some embodiments, the ligase includes an enzyme capable of catalyzing the joining of nicks between adjacent nucleotides of a nucleic acid. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases can include, but not limited to, T4 DNA ligase, T4 RNA ligase, and E. coli DNA ligase.

As used herein, “blunt-end ligation” and its derivatives, refers generally to ligation of two blunt-end double-stranded nucleic acid molecules to each other. A “blunt end” refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion greater than two nucleotides in length, referred to herein as an “overhang.” In some embodiments, the end of nucleic acid molecule does not include any single stranded portion, such that every nucleotide in one strand of the end is based paired with opposing nucleotides in the other strand of the same nucleic acid molecule. In some embodiments, the ends of the two blunt ended nucleic acid molecules that become ligated to each other do not include any overlapping, shared or complementary sequence. In some embodiments, blunt-ended ligation includes a nick translation reaction to seal a nick created during the ligation process.

As used herein, “ligation conditions” and its derivatives, generally refers to conditions suitable for ligating two molecules to each other. In some embodiments, the ligation conditions are suitable for sealing nicks or gaps between nucleic acids. As defined herein, a “nick” or “gap” refers to a nucleic acid molecule that lacks a directly bound 5′ phosphate of a mononucleotide pentose ring to a 3′ hydroxyl of a neighboring mononucleotide pentose ring within internal nucleotides of a nucleic acid sequence. As used herein, the term nick or gap is consistent with the use of the term in the art. Typically, a nick or gap can be ligated in the presence of an enzyme, such as ligase at an appropriate temperature and pH. In some embodiments, T4 DNA ligase can join a nick between nucleic acids at a temperature of about 70° C.-72° C. In some embodiments, the ligation reaction is performed at a final pH of 8-9, pH 8-8.7, pH 8-8.5, or any final pH or ranges therein. In some embodiments, the ligation reaction is performed at a final PEG concentration of 4% to less than 10%, 4% to 8%, 4% to 6%, or any final PEG concentrations or ranges therein. One of skill in the art can determine the pH and PEG % in the ligation buffer needed to achieve the pH and PEG % in the ligation reaction as disclosed herein. In some embodiments, by increasing the pH as disclosed herein, the PEG % can be lowered as disclosed herein in the ligation mix for efficient automation and increased reproducibility.

As used herein, “polymerase” and its derivatives, generally refers to any enzyme that can catalyze the polymerization of nucleotides (including analogs thereof) into a nucleic acid strand. Typically, but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated. In some embodiments, the polymerase is an antibody based thermostable polymerase, such as an antibody based hot-start polymerase. Such polymerases are readily available commercially.

The term “extension,” “extending,” and its variants, as used herein, when used in reference to a given primer, comprises any in vivo or in vitro enzymatic activity characteristic of a given polymerase that relates to polymerization of one or more nucleotides onto an end of an existing nucleic acid molecule, to generate an extension product. Typically, but not necessarily, such primer extension occurs in a template-dependent fashion; during template-dependent extension, the order and selection of bases is driven by established base pairing rules, which can include Watson-Crick type base pairing rules or alternatively (and especially in the case of extension reactions involving nucleotide analogs) by some other type of base pairing paradigm. In one non-limiting example, extension occurs via polymerization of nucleotides on the 3′OH end of the nucleic acid molecule by the polymerase.

The term “hybridize” refers to a sequence specific non-covalent binding interaction with a complementary nucleic acid. Hybridization can occur to all or a portion of a nucleic acid sequence. Those skilled in the art will recognize that the stability of a nucleic acid duplex, or hybrids, can be determined by the Tm. Additional guidance regarding hybridization conditions can be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, Vol. 3, 1989.

As used herein, “incorporating” a sequence into a polynucleotide refers to covalently linking a series of nucleotides with the rest of the polynucleotide, for example at the 3′ or 5′ end of the polynucleotide, by phosphodiester bonds, wherein the nucleotides are linked in the order prescribed by the sequence. A sequence has been “incorporated” into a polynucleotide, or equivalently the polynucleotide “incorporates” the sequence, if the polynucleotide contains the sequence or a complement thereof. Incorporation of a sequence into a polynucleotide can occur enzymatically (e.g., by ligation or polymerization) or using chemical synthesis (e.g., by phosphoramidite chemistry).

The term “associated” is used herein to refer to the relationship between a sample and the DNA molecules, RNA molecules, or other polynucleotides originating from or derived from that sample. A polynucleotide is associated with a sample if it is an endogenous polynucleotide, i.e., it occurs in the sample at the time the sample is selected or is derived from an endogenous polynucleotide. For example, DNAs endogenous to a cell are associated with that cell. cDNAs resulting from reverse transcription of mRNAs, and DNA amplicons resulting from PCR amplification of the cDNAs, contain the sequences of the mRNAs and are also associated with the cell. The polynucleotides associated with a sample need not be located or synthesized in the sample and are considered associated with the sample even after the sample has been destroyed (for example, after a cell has been lysed). Molecular barcoding or other techniques can be used to determine which polynucleotides in a mixture are associated with a particular sample.

When hybridization occurs in an antiparallel configuration between two single-stranded polynucleotides, the reaction is called “annealing” and those polynucleotides are described as “complementary”. As used herein, and unless otherwise indicated, the term “complementary,” when used to describe a first nucleotide sequence in relation to a second nucleotide sequence, refers to the ability of a polynucleotide comprising the first nucleotide sequence to hybridize and form a duplex structure under certain conditions with a polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences include base-pairing of a region of a polynucleotide comprising a first nucleotide sequence to a region of a polynucleotide comprising a second nucleotide sequence over the length or a portion of the length of one or both nucleotide sequences. Such sequences can be referred to as “complementary” with respect to each other herein. However, where a first sequence is referred to as “substantially complementary” with respect to a second sequence herein, the two sequences can be complementary, or they can include one or more, but generally not more than about 5, 4, 3, or 2 mismatched base pairs within regions that are base-paired. For two sequences with mismatched base pairs, the sequences will be considered “substantially complementary” as long as the two nucleotide sequences bind to each other via base-pairing.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′-end of the coding RNA transcript are referred to as “downstream sequences.”

In some embodiments, the double stranded target DNA molecules can be end repaired so that they are amenable for ligation. For example, the ends of the target DNA molecules can be polished to have blunt ends. As known in the art, this can be achieved with enzymes that can either fill in or remove the protruding strand. The ends of the target DNA molecules can also be adenylated, e.g., at the 3′ end by a T4 polymerase.

In the methods disclosed herein, synthetic oligonucleotides, called “adapter(s),” can be ligated with one or both termini (5′ and 3′) of the target DNA molecules. In some embodiments, the adapter(s) are useful in a sequencing platform. The adapters can comprise one or more deoxyuridines (U, dU, or dUMP), such as 1, 2, 3, 4, 6, or more and a unique molecular index (UMI). The adapters can be double stranded, or single stranded and then extended to double stranded by extension. In some embodiments, the adapters do not comprise a sample index. In some embodiments, adapters can comprise one or more dUs and UMI.

In some embodiments, the adapter is a single adapter that is annealed to the target DNA molecule and extended, wherein the adapter comprises a UMI and a target specific sequence that does not contain a deoxyuridine. As used herein, a “single” adapter means that one adapter is annealed to the target DNA molecule but of course, multiple target DNA molecules can be annealed with single adapters simultaneously. In other embodiments, a pair of adapters are annealed to the target DNA molecule (which is double stranded) and extended, wherein each adapter of the pair of adapters comprises a UMI, a first or second target specific sequence, and one or more deoxyuridines.

A target specific sequence is a sequence in the adapter or primer, as specified, that can hybridize to a target sequence (e.g., a known or predetermined sequence) on a target DNA molecule so that adjacent sequence on the target DNA molecule can be determined.

Unique molecular indices or identifiers (UMIs; also called Random Molecular Tags (RMTs)) are short sequences or “barcodes” of bases used to tag each DNA molecule (fragment) prior to library amplification, thereby aiding in the identification of each individual nucleic acid molecule, or PCR duplicates. Kivioja, T. et al., Nat. Methods 9:72-74 (2012), and Suppl. If two reads align to the same location and have the same UMI, it is highly likely that they are PCR duplicates originating from the same fragment prior to amplification.

The concept of UMIs is that prior to any amplification, each original target molecule is ‘tagged’ by a unique barcode sequence. This DNA sequence must be long enough to provide sufficient permutations to assign each founder molecule a unique barcode. In some embodiments, a UMI sequence contains randomized nucleotides and is incorporated into the adapter. For example, a 12-base random sequence provides 412 or 16,777,216 UMI's for each target molecule or DNA fragment in the sample.

After adapters are ligated or annealed to the target DNA molecules, unused adapters can be removed by treatment with UDG (uracil DNA glycosylase). UDG treatment can also be performed to remove unused target specific primers containing U base(s). UDG cleaves uracil bases from DNA. Thus, UDG treatment can also cleave the U base(s) in the DNA strand with the adapter containing U or dU base(s), leaving intact the copied strand contain the A or dA base(s). UDG can be from prokaryotes or eukaryotes, such as but not limited to UNG, SMUGI, TDG, and MBD4.

The methods disclosed herein can further comprise amplifying the target DNA molecules for enrichment. Target enrichment can be achieved with, e.g., an SPE primer or an SPE primer pool. Amplicon-based next-generation sequencing (NGS) assays offer many advantages for targeted enrichment. For example, QIAseq NGS panels employ unique molecular indices (UMI's) to correct for PCR amplification bias and use single primer extension (SPE) technology which provides design flexibility and highly specific target enrichment.

As used herein, the term “primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually, primers are extended by a DNA polymerase. Primers usually have a length in the range of between 3 to 36 nucleotides, also 5 to 24 nucleotides, also from 14 to 36 nucleotides. Primers as disclosed herein include target specific primers, universal primers, amplification primers and the like. Primers and probes can be degenerate in sequence. Primers as disclosed herein can bind adjacent to a sequence to be determined. A “primer” can be considered a short polynucleotide, generally with a free 3′—OH group that binds to a target nucleic acid molecule or template potentially present in a sample of interest by hybridizing to the target sequence, and thereafter promoting polymerization of a polynucleotide complementary to the target nucleic acid molecule. Primers used in the methods disclosed herein can be comprised of nucleotides ranging from 17 to 30 nucleotides. In some embodiments, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides. In some embodiments, a single stranded adapter can act as a primer for extension.

As used herein, “target specific primer” and its derivatives, refers to a primer that targets a target DNA molecule. A target specific primer generally binds or hybridizes to a single-stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or 100% complementary, to at least a portion of a target nucleic acid molecule that includes a target sequence and an adjacent sequence to be determined. The target specific primer can contain a sequence that is complementary to a region of a target molecule that contain the entire or a portion of a target sequence. When the target specific primer contains a sequence that is complementary to a target sequence, the target specific primer and target sequence are described as “corresponding” to each other. In some embodiments, the target specific primer is capable of hybridizing to at least a portion of its corresponding target sequence (or to a complement of the target sequence); such hybridization can optionally be performed under standard hybridization conditions or under stringent hybridization conditions.

In some embodiments, the target specific primer is substantially non-complementary to other target sequences present in the target DNA molecule or sample; optionally, the target specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target specific primer is designed to include a nucleotide sequence that is substantially complementary to at least a portion of its corresponding target sequence. In some embodiments, a target specific primer is at least 95% complementary, or at least 99% complementary, or 100% identical, across its entire length to at least a portion of a target nucleic acid molecule that includes its corresponding target sequence. In some embodiments, a target specific primer that is complementary includes at least 90%, at least 95% complementary, at least 98% complementary or at least 99% complementary, or 100% complementary, across its entire length to at least a portion of its corresponding target sequence in the target nucleic acid molecule. In some embodiments, the target specific primer can be substantially non-complementary at its 3′ end or its 5′ end to any other target specific primer present in an amplification reaction. In some embodiments, the target specific primer can include minimal cross hybridization to other target specific primers in the amplification reaction. In some embodiments, target specific primers include minimal cross-hybridization to non-specific sequences in the amplification reaction mixture. In some embodiments, the target specific primers include minimal self-complementarity. In some embodiments, the target specific primers can include one or more cleavable groups located at the 3′ end.

In some embodiments, the target specific primers can include one or more cleavable groups located near or about a central nucleotide of the target specific primer. In some embodiments, one of more targets specific primers includes only non-cleavable nucleotides at the 5′ end of the target specific primer. In some embodiments, a target specific primer includes minimal nucleotide sequence overlap at the 3′end or the 5′ end of the primer as compared to one or more different target specific primers, optionally in the same amplification reaction. In some embodiments 1, 2, 3, 4, 5, 6, 7, 8, 9, 10 or more, target specific primers in a single reaction mixture include one or more of the above embodiments. In some embodiments, substantially all of the plurality of target specific primers in a single reaction mixture includes one or more of the above embodiments.

In some embodiments, target specific primers can further comprise a tag sequence. For example, a target specific primer can comprise a tag sequence comprising at least 2 dUs and a target specific sequence comprising 1, 2, 3, or 4 dUs in a 5′ to 3′ direction.

A “tag sequence” refers to a universal sequence attached to the 5′ of the target specific primer. This 5′ tag sequence is a binding site for the universal primer.

The methods disclosed herein enables the use of small amounts of nucleic acids. For example, isolated DNA amounts of 0.5 ng to 100 ng, 1 ng to 75 ng, 5 ng to 50 ng, 10 ng to 20 ng, or any amounts or ranges derived therefrom can be fragmented and used for the library construction disclosed herein.

In some embodiments, the target specific primers that are used for single primer extension can be designed with a mean or median melting temperature (Tm) of 65° C. to 70° C., 66° C. to 69° C., or 67° C. to 68° C., or any specific temperature or ranges derived therefrom, e.g., 67.6° C. The Tm primer design can confer high specificity during SPE enrichment PCR with only a single target specific primer.

As disclosed herein, target specific primer design can be based on single primer extension, in which each genomic target is enriched by one target specific primer and one universal primer—a strategy that removes conventional two target specific primer design restriction and reduces the amount of the required primers. All primers required for a panel are pooled into an individual primer pool to reduce panel handling and the number of pools required for enrichment and library construction.

The booster panel is a pool of up to 100 primers that can be used to boost the performance of certain primers in any panel (cataloged, extended, or custom), or to extend the contents of an existing custom panel. The primers are delivered as a single pool that can be spiked into the existing panel.

After removing unused adapters or unused target specific primers comprising dU(s) by treatment with UDG, a limited number of PCR cycles can be conducted using a pool of single target specific primers, each carrying a target specific sequence complementary to the gene or loci of interest and a 5′ universal sequence. During this process, each single target specific primer repeatedly samples the same target locus from different DNA templates or target nucleic acid molecules. Afterwards, additional PCR cycles can be conducted using universal primers to amplify the library to the desired quantity.

Compared to existing targeted enrichment approaches, the SPE method can be based on single end adapter ligation, which has higher efficiency than requiring adapters to ligate to both ends of the dsDNA fragment. More DNA molecules will be available for the downstream PCR enrichment step. PCR enrichment efficiency using one target specific primer is also better than conventional two target specific primer approach, due to the absence of an efficiency constraint from a second primer. During the initial PCR cycles, primers have repeated opportunities to convert (i.e., capture) maximal amount of original DNA molecules into amplicons.

All these features help to increase the efficiency of capturing rare mutations in the sample. In addition, incorporated UMI's within the amplicon are the key to estimating the number of DNA molecules captured and to greatly reduce sequencing errors in downstream analysis. Single primer extension also permits discovery of unknown structural variants, such as gene fusions.

The methods disclosed herein can further comprise amplifying the target amplification product with a universal primer to generate a second target amplification product. A “universal primer” comprises a sequence complementary to a tag sequence or nucleotide sequences that are very common in a particular set of DNA molecules and cloning vectors. Thus, universal primers are able to bind to a wide variety of DNA templates. In the methods disclosed herein, the universal primer also comprises a sample index but does not comprise any deoxyuridines. A “sample index” comprises a unique sequence that identifies one sample so that multiple samples can be mixed and sequenced at the same time.

As used herein, the terms “amplify” and “amplification” refer to enzymatically copying the sequence of a polynucleotide, in whole or in part, so as to generate more polynucleotides that also contain the sequence or a complement thereof. The sequence being copied is referred to as the template sequence. Examples of amplification include DNA-templated RNA synthesis by RNA polymerase, RNA-templated first-strand cDNA synthesis by reverse transcriptase, and DNA-templated PCR amplification using a thermostable DNA polymerase Amplification includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

The terms “PCR product,” “PCR fragment,” “amplification product,” and “amplicon” refer to the resultant mixture of compounds after two or more cycles of the PCR steps of denaturation, annealing and extension are complete. These terms encompass the case where there has been amplification of one or more segments of a target nucleic acid molecule(s), i.e., target amplification product.

As used herein, “target amplification product” or “amplified target sequences” and its derivatives, refers generally to a nucleic acid sequence produced by the amplification of/amplifying the target nucleic acid molecule using target specific primers (primers comprising a target specific sequence that hybridizes to a target sequence) and the methods provided herein. The amplified target sequences can be either of the same sense (the positive strand produced in the second round and subsequent even-numbered rounds of amplification) or antisense (i.e., the negative strand produced during the first and subsequent odd-numbered rounds of amplification) with respect to the target sequences. For the purposes of this disclosure, the amplified target sequences are typically less than 50% complementary to any portion of another amplified target sequence in the reaction.

The term “polymerase chain reaction” (“PCR”) of Mullis (U.S. Pat. Nos. 4,683,195, 4,683,202, and 4,965,188) refers to a method for increasing the concentration of a segment of a target nucleic acid molecule in a mixture of nucleic acid molecules without cloning or purification. This process for amplifying the target nucleic acid molecule comprises introducing a large excess of two oligonucleotide primers to the nucleic acid mixture containing the desired target nucleic acid molecule, followed by a precise sequence of thermal cycling in the presence of a polymerase (e.g., DNA polymerase). The two primers are complementary to their respective strands of the double stranded target nucleic acid molecule. To effect amplification, the mixture is denatured and the primers then annealed to their complementary sequences within the target nucleic acid molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle;” there can be numerous “cycles”) to obtain a high concentration of an amplified segment of the desired target nucleic acid molecule. The length of the amplified segment of the desired target nucleic acid molecule is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target nucleic acid molecule become the predominant nucleic acid molecules (in terms of concentration) in the mixture, they are said to be “PCR amplified.”

A real-time polymerase chain reaction (Real-Time PCR), also known as quantitative polymerase chain reaction (qPCR), is a laboratory technique of molecular biology based on the polymerase chain reaction (PCR). It monitors the amplification of a targeted DNA molecule during the PCR, i.e. in real-time, and not at its end, as in conventional PCR. Real-time PCR can be used quantitatively (quantitative real-time PCR), and semi-quantitatively, i.e., above/below a certain amount of DNA molecules (semi quantitative real-time PCR). Other types of PCRs include but are not limited to nested PCR (used to analyze DNA sequences coming from different organisms of the same species but that can differ for a single nucleotide (SNIPS) and to ensure amplification of the sequence of interest in each of the organism analyzed) and Inverse-PCR (usually used to clone a region flanking an insert or a transposable element).

Two common methods for the detection of PCR products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary sequence.

“Multiplex amplification” refers to simultaneous amplification of more than one target nucleic acid molecule in one reaction vessel. In some embodiments, methods involve subsequent determination of the sequence of the multiplex amplification products using one or more sets of primers. Multiplex can refer to the detection of between about 2-1,000 different sequences of interest in a single reaction. As used herein, multiplex refers to the detection of any range between 2-1,000, e.g., between 5-500, 25-1000, or 10-100 different sequences of interest in a single reaction, etc. The term “multiplex” as applied to PCR implies that there are primers specific for at least two different sequences of interest or two or more different regions of the same sequence of interest in the same PCR reaction. In embodiments of methods described herein, multiplex applications can include determining the nucleotide sequence contiguous to one or more known target nucleotide sequences in multiple samples in one sequencing reaction or sequencing run. In some embodiments, multiple samples can be of different origins, e.g., from different tissues and/or different subjects. In some embodiments, a primer (e.g., an adapter) with a unique barcode can be added to each molecule and ligated to the nucleic acids therein; the samples can subsequently be pooled. In such embodiments, each resulting sequencing read of an amplification product will comprise a barcode that identifies the original nucleic acid molecule or template nucleic acid from which the amplification product is derived.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nucl. Acids Res., 28, e63, 2000), each of which is hereby incorporated by reference in its entirety.

Reagents and hardware for conducting amplification reaction are commercially available. Primers useful to amplify sequences from a particular gene region are preferably complementary to and hybridize specifically to sequences in the target region or in its flanking regions and can be prepared using the polynucleotide sequences provided herein. Nucleic acid sequences generated by amplification can be sequenced directly.

Methods and kits for performing PCR are well known in the art. PCR is a reaction in which replicate copies are made of a target polynucleotide using a pair of primers or a set of primers consisting of an upstream and a downstream primer, and a catalyst of polymerization, such as a DNA polymerase, and typically a thermally stable polymerase enzyme. Methods for PCR are well known in the art, and taught, for example in MacPherson et al. (1991) PCR 1: A Practical Approach (IRL Press at Oxford University Press).

In the methods disclosed herein, by using adapters and target specific primers comprising one or more deoxyuridines, the adapter ligation and/or extension products and target amplification products can be cleaned up with UDG, without the use of multiple bead purification. A final bead purification can be used to prepare the DNA library for analysis.

The DNA library prepared by the methods disclosed herein can be sequenced and analyzed using methods known to those of skill in the art, e.g., by next-generation sequencing (NGS). In certain exemplary embodiments, RNA expression profiles are determined using any sequencing methods known in the art. Determination of the sequence of a nucleic acid sequence of interest can be performed using a variety of sequencing methods known in the art including, but not limited to, sequencing by synthesis (SBS), sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (US2009/0018024), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLID, Helicos, Complete Genomics, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

Disclosed herein are methods for analyzing gene expression in a plurality of single cells, the method comprising the steps of preparing a cDNA library using the method described herein and sequencing the cDNA library. A “gene” refers to a polynucleotide containing at least one open reading frame (ORF) that is capable of encoding a particular polypeptide or protein after being transcribed and translated. Any of the polynucleotide sequences described herein can be used to identify larger fragments or full-length coding sequences of the gene with which they are associated. Methods of isolating larger fragment sequences are known to those of skill in the art.

The DNA library can be sequenced by any suitable screening method. In particular, the DNA library can be sequenced using a high-throughput screening method, such as Applied Biosystems' SOLiD sequencing technology, or Illumina's Genome Analyzer. In one aspect of the invention, the DNA library can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

The DNA libraries generated by the methods disclosed herein can be useful for, but not limited to, DNA variant detection, copy number analysis, fusion gene detection and structural variant detection. In some embodiments, the DNA library can be used for next generation sequencing, profiling of DNA variants, human identity or paternity testing, pain or ADME pharmacogenomics, or detection of a genetic disease.

### EXAMPLES

### Example 1. Workflow

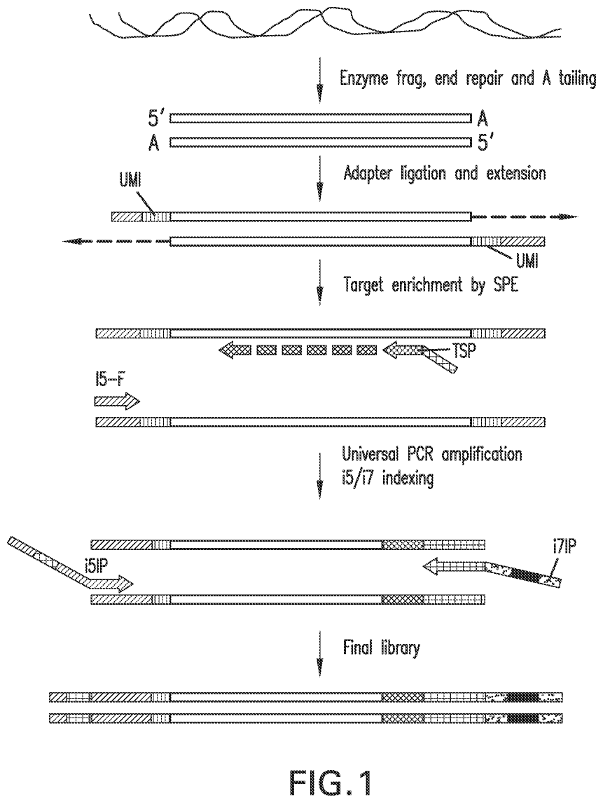

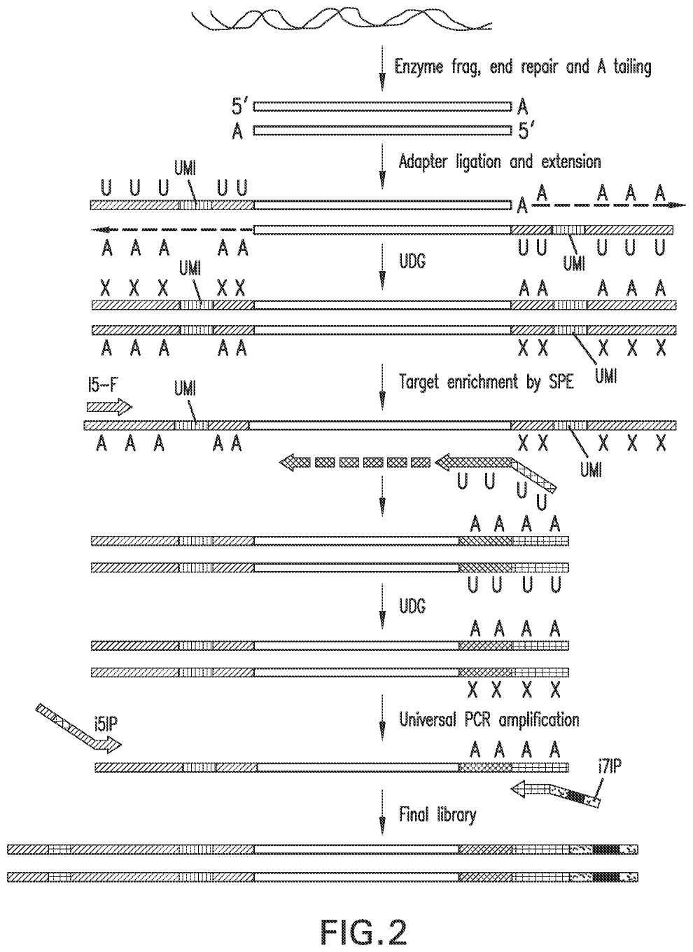

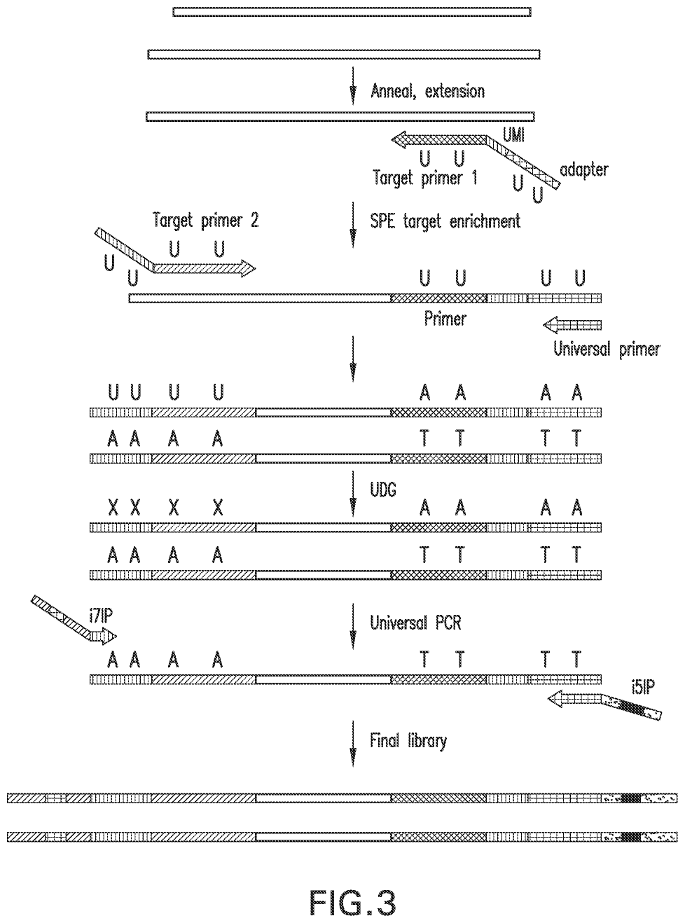

The entire workflow, as exemplified in FIG. 1, 2, or 3, from extracted DNA to sequencing-ready libraries can be completed in 9 hours. Extracted DNA is fragmented, genomic targets are molecularly barcoded and enriched, and libraries are constructed. Sequencing files can be fed into the QIAseq pipeline, a cloud-based data analysis pipeline, which will filter, map, and align reads, as well as count unique molecular barcodes associated with targeted genomic regions, and call variants with a barcode-aware algorithm. This data can then be fed into IVA or QCI for interpretation.

### Example 2. Adapters and Primers

Isolated DNA, as low as 0.5 ng, e.g., from 0.5 ng to 100 ng, is enzymatically fragmented to generate small pieces of dsDNA. This is followed by library construction, in which adapters, molecular barcode, and sample indexes are incorporated into the DNA fragments. Library fragments now serve as templates for target enrichment using single primer extension. The targets are enriched using a universal forward primer (e.g., 15-F forward primer) and a single target specific primer. Finally, library amplification and sample indexing (for dual indexing) use the 15IP and 17IP sample index primers. Example adapter and primer sequences are provided below.

Adapter (SEQ ID NO: 1):
5′-CG/ideoxyU/CGGCAGCG/ideoxyU/CAGATGTGTA/
ideoxyU/AAGAGACAGNNNNNNNNNNNNATGCA/ideoxyU/
TCGAG/ideoxyU/CA*T-3′
15-F forward primer (SEQ ID NO: 2):
5′-CGTCGGCAGCGTCAGATGTGTATAAGAGACAG-3′
Target specific primer (SEQ ID NO: 3):
5′-AATGTACAG/ideoxyU/ATTGCGTTT/ideoxyU/
G-target specific (average 35 nt, 0-2
ideoxyU)-3′
15IP index primer
(with one sample index as example)
(SEQ ID NO: 4):
5′-AATGATACGGCGACCACCGAGATCTACACATGGCCGA
CTTCGTCGGCAGCGTCAGATGTGTATAAGAGAC*A-3′
I7IP index primer (with one sample
index as example) (SEQ ID NO: 5):
5′-CAAGCAGAAGACGGCATACGAGATTGAACGTTGTGTGA
CTGGAGTTCAGACGTGTGCTCTTCCGATCTCCAG+TAC+AG
T+ATTGCGTTTT*G-3′
“ideoxyU” denotes internal deoxyuridine.
“*” denotes a phosphorothioate bond. When
placed between the last and second to last
bases, the oligo is less susceptible to
potential exonuclease activity.
“+” denotes a locked nucleic acid (LNA).
LNA is a modified RNA nucleotide in which
the ribose moiety is modified with an
extra bridge connecting the 2′ oxygen
and 4′ carbon and is used to increase
the Tm of oligo binding.
“I5” and “17” denote Illumina adapters.

### Example 3. Ligation

Automation of library construction is critical for high throughput labs. Virtually all library protocols require ligation of adapters and high efficiency adapter ligation requires high levels of PEG (extremely viscous). A new reaction buffer that allows trace amounts of PEG to be used facilitates simpler automation.

During NGS library construction, high efficiency of adapter ligation is critical and is usually achieved by using high amount of PEG (10% or above) during ligation reaction. This makes ligation reaction extremely viscous and is difficult for both manual and automation handling. A novel ligation buffer is developed by increasing buffer pH from regular 7.6 to 8 or above. This high pH ligation buffer could achieve similar ligation efficiency with relative low PEG (6%) compared to ligation with higher PEG amount. In addition, the high pH buffer was made at 2.5× compared to the 5× of pH 7.6 buffer, this decreases the viscosity of ligation buffer itself.

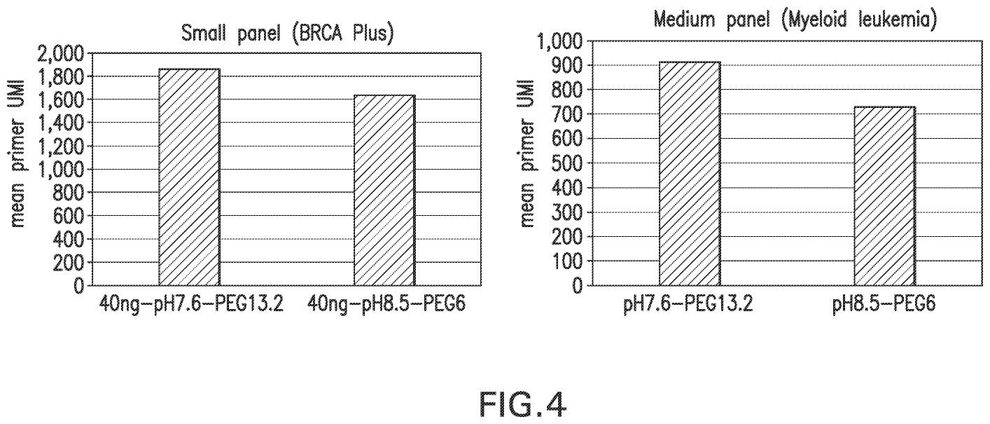

The initial test of high pH ligation buffer was done on the current QIAseq Targeted DNA panel workflow (V3). The ligation buffer of pH 8.5 with final 6% PEG in ligation was compared to the buffer of pH 7.6 with final 13.2% PEG, extra PEG was added to ligation reaction with pH 7.6 buffer to reach the final 13.2% PEG in the reaction. Forty ng of NA12878 DNA was used to construct library according to QIAseq Targeted DNA panel workflow handbook with two ligation buffers of different pH and final PEG concentration in the reaction. After adapter ligation, a small and a medium size of DNA panel were used to do target enrichment. The small panel was QIAseq Targeted DNA BRCA Plus panel with 348 primers while the medium panel was Myeloid leukemia panel of 5887 primers. Final libraries were sequenced on Miseq for small panel and NextSeq for medium panel, recovered UMI numbers were used to access the ligation efficiency between two buffers. From the result, ligation buffer of pH 8.5 with 6% PEG in the reaction had similar or slightly lower UMI compared to ligation buffer of pH 7.6 with 13.2% PEG (FIG. 4), suggesting high pH ligation buffer of low PEG in the reaction could achieve similar ligation efficiency as regular pH ligation buffer with higher PEG in the reaction.

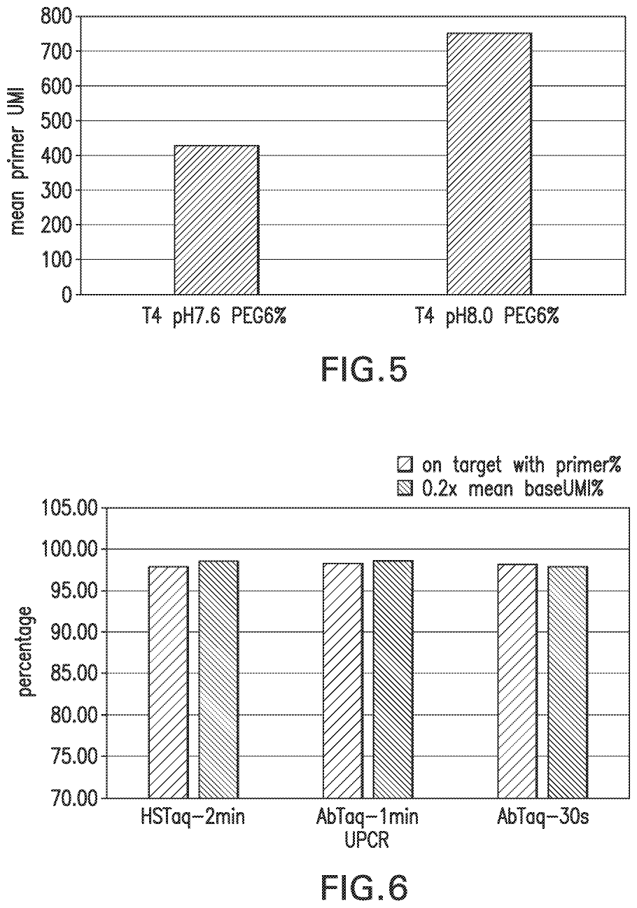

This high pH ligation buffer was further evaluated in V4 DNA panel workflow by comparing pH 8.0 ligation buffer with pH 7.6 ligation buffer, both had 6% PEG in the final ligation reaction. 10 ng NA12878 DNA was used as input DNA and a custom V4 panel with 480 primers was used for target enrichment PCR. Ligation efficiency was checked by UMI numbers after sequencing. The ligation buffer with high pH had much more UMI compared to the buffer with low pH suggesting the high pH ligation buffer can be used in V4 DNA panel workflow also (FIG. 5).

Formulation of ligation buffers are provided below in Tables A and B.

TABLE A
Regular 5x ligation buffer of pH 7.6

| Component | Final Conc of 5X Buffer | |
|---|---|---|
| Tris-HCl | 330 | mM |
| Magnesium Chloride | 50 | mM |
| PEG-6000 | 30% | |
| ATP | 5 | mM |
| DTT | 5 | mM |

pH to 7.6

TABLE B
2.5x ligation buffer of pH 8.0 or 8.5

| Component | Final Conc of 2.5X Buffer | |
|---|---|---|
| Tris-HCl pH 8.0 | 165 | mM |
| Magnesium Chloride | 25 | mM |
| PEG-6000 | 15% | |
| ATP | 2.5 | mM |
| DTT | 2.5 | mM |

pH to 8.0 or 8.5

### Example 4. Fast Cycling

In order to enable additional time savings (a shortcoming of most library protocols is long construction time) a much faster cycling Ab hotstart taq is used. The challenge is that high complexity library construction is extraordinarily dependent on the enzyme and conditions used for library amplification. The initial optimization of V3 chemistry took months. Here, this critical step was replaced with one that will provide equivalent performance, can cycle fast, and tolerate the dU modifications required in the primers and adapters. This successful improvement led to savings of hours of time in library prep.

A faster cycling Ab hotstart taq was used in both target enrichment PCR and universal PCR to enable much quicker cycling compared to the current V3 PCR reaction.

To further decrease the workflow time, Ab hotstart Taq was also tested in the final universal PCR. The universal PCR was carried out either with chem hotstar Taq for 2 min at 62C anneal/extension in each cycle or with Ab hotstart Taq for 1 min or 30 sec at 62° C. anneal/extension. General panel performance of specificity and uniformity looked similar among these conditions (FIG. 6). Therefore, Abhotstart Taq could be used in universal PCR with 30 sec anneal/extension and this decreased universal PCR time from previously about 1.5 hrs to 45 min.

### Example 5. Bead Cleanup

Bead cleanups using SPRI beads between steps in NGS library construction serve two functions: removal of unincorporated primers and adapters, which would both interfere with downstream steps and (in the case of UMI containing adapters) cause re-barcoding of library clones, rendering the UMIs essentially useless; and size selection of the library.

Manipulation of the PEG concentration when binding a library to beads enables the selective loss of either shorter-than-desired DNA (e.g., primer dimers or very small library fragments) or long DNA (which may not effectively contribute to functional library molecules).

Bead cleanup is fraught with problems. First, a bead cleanup of the primary sample after ligation causes the unavoidable loss of unique original molecules, reducing the complexity of the library. Second. The bead cleanups are the major cause of variability in yield, presence of primer dimers, and contamination with other unwanted sequences, due to sample to sample variations in users' hands even if automated. It is often possible to look at final sequencing metrics and know which technician made the library based on differences in bead handling techniques. The current invention allows the replacement of all but a final perfunctory bead cleanup to be replaced with a simple, reliable enzymatic reaction. The result is higher yield, more reproducible libraries, ease of automation, and significant time savings.

### Example 6. Protocol

Important points before starting. This protocol covers all procedures required for the preparation of libraries for Illumina sequencers from “standard DNA” (i.e., cells or tissues), FFPE DNA and cfDNA. Before setting up the reaction, it is critical to accurately determine the amount of the input DNA (10-80 ng for standard DNA or cfDNA; up to 250 ng of FFPE DNA can be used, if QIAseq QuantiMIZE kits have been used. If an alternative method was used to determine the concentration of FFPE DNA, then up to 100 ng DNA can be used). Lower input amounts are possible; however, this will lead to fewer sequenced UMIs and reduced variant detection sensitivity. To reach certain variant detection level, both DNA input amount and sequencing depth are required (Table 1). QIAseq Beads are used for final reaction cleanups. Important: Prepare fresh 80% ethanol daily. Reaction and cleanup procedures can be performed in either PCR tubes or 96-well plate. Upon completion of the library preparation, the QIAseq Library Quant System can be used for library quantification.

TABLE 1
Suggested fresh DNA input amount and sequencing
depth for variant detection*

| Variant frequency | Input (ng) | Read pairs/UMI | Mean read |
|---|---|---|---|
| 5% | 10 | 4 | 7200 |
| 1% | 40 | 4 | 25600 |

*Variant detection is based on 90% sensitivity on the entire region of QIAseq Targeted DNA Pro Panel.

### FFPE DNA Repair.

For FFPE DNA, set up a repair reaction first before doing “Fragmentation, end-repair and A-addition”. For standard and cfDNA, go directly to “Fragmentation, end-repair and A-addition” step.

On ice, prepare the FFPE DNA repair reaction mix according to Table 2. Briefly centrifuge, mix by pipetting up and down 7-8 times and briefly centrifuge again. Note: In general, increasing the amount of FFPE DNA input will improve variantdetection sensitivity. Important: Keep the reaction tubes/plate on ice during the entire reactionsetup.

TABLE 2
Reaction mix for FFPE DNA repair

| Component | Volume/reaction | |
|---|---|---|
| FFPE DNA* | Variable | |
| 10X Fragmentation Buffer | 1.4 | μl |
| FFPE Repair reagent | 0.6 | μl |
| FFPE Repair enzyme | 1 | μl |
| Nuclease-Free Water | Variable | |
| Total | 12 | μl |

*Use up to 250 ng of FFPE DNA if QIAseq QuantiMIZE kits were used, or up to 100 ng of FFPE DNA if an alternative method was used.

Program the thermal cycler according to Table 3. Use the instrument's heated lid. Before adding the tubes/plate to a thermal cycler, start the program. When thethermal cycler reaches 4° C., pause the program. Transfer the tubes/plate prepared in step 2 to the pre-chilled thermal cycler andresume the cycling program.

TABLE 3
Cycling conditions for FFPE DNA repair reaction

| | | | Incubation | | Incubation | |
|---|---|---|---|---|---|---|
| | | | time (thermal | | time (thermal | |
| | Incubation | | cycler without | | cycler with | |
| Step | temperature | | ramping control) | | ramping control) | |
| 1 | 4° | C. | 2 | min | 2 | min |
| 2 | 15° | C. | 30 | sec | 0.1° C./sec from 4° C. to 37° C. | |
| | 25° | C. | 30 | sec | | |
| 3 | 37° | C. | 30 | min | 30 | min |

| | | | | |
|---|---|---|---|---|
| 4 | 4° | C. | Hold | Hold |

After reaction finished, place the samples on ice and immediately proceed with “Fragmentation, end-repair and A-addition” below.

### Fragmentation, End-Repair and A-Addition.

On ice, prepare the fragmentation, end-repair and A-addition mix according to Table 4. Briefly centrifuge, mix by pipetting up and down at least 10-12 timesand briefly centrifuge again. Note: In general, increasing the amount of DNA input will improve variantdetection sensitivity. Important: Keep the reaction tubes/plate on ice during the entire reactionsetup.

TABLE 4
Reaction mix for fragmentation, end-repair and A-addition

| | Volume/reaction | | Volume/reaction | | Volume/reaction | |
|---|---|---|---|---|---|---|
| Component | (standard DNA) | | (FFPE DNA) | | (cfDNA) | |
| DNA* | Variable | | — | | Variable | |
| 10X | 1.4 | μl | — | | 1.4 | μl |
| Fragmentation | | | | | | |
| Buffer | | | | | | |
| 5X WGS | 2.8 | μl | 2.8 | μl | 2.8 | μl |
| Fragmentation | | | | | | |
| Mix | | | | | | |
| FG Solution | — | | — | | 1.4 | μl |
| Nuclease-Free | Variable | | Variable | | Variable | |
| Water | | | | | | |
| Total | 14 | μl | 14.8 | μl | 14 | μl |

*10-80 ng for standard DNA or cfDNA. Use up to 250 ng of FFPE DNA if QIAseq QuantiMIZE kits were used, or up to 100 ng of FFPE DNA if an alternative method was used.

Program the thermal cycler according to Table 5. Use the instrument's heated lid. Before adding the tubes/plate to a thermal cycler, start the program. When thethermal cycler reaches 4° C., pause the program. Important: The thermal cycler must be pre-chilled and paused at 4° C. Transfer the tubes/plate prepared in step 7 to the pre-chilled thermal cycler andresume the cycling program.

TABLE 5
Cycling conditions for fragmentation, end-repair and A-addition

| | | |
|---|---|---|
| Step | Incubation temperature | Incubation time |

| | | | | |
|---|---|---|---|---|
| 1 | 4° | C. | 1 | min |
| 2 | 32° | C. | 10 | min |
| 3 | 65° | C. | 15 | min |

| | | | |
|---|---|---|---|
| 4 | 4° | C. | Hold |

Upon completion, allow the thermal cycler to return to 4° C. Place the samples on ice and immediately proceed with “Adapter ligation”, below.

### Adapter Ligation.

Prepare the adapter ligation mix according to Table 6. Briefly centrifuge, mix by pipetting up and down 10-12 times and briefly centrifuge again. Important: Adapter does not contain sample index, one single adapter is usedfor all samples.

TABLE 6
Reaction mix for adapter ligation

| Component | Volume/reaction | |
|---|---|---|
| Fragmentation, end-repair and | 14 | μl |
| A-addition reaction (already in tube) | | |
| UPH Ligation Buffer, 2.5x | 10 | μl |
| AdP-IL5-Phased Adapter | 1.5 | μl |
| DNA Ligase | 2.5 | μl |
| Nuclease-Free Water | — | |
| Total | 28 | μl |

Program the thermal cycler according to Table 7. Important: Do not use heated lid during 20° C. stage. Before adding the tubes/plate to a thermal cycler, start the program. When thethermal cycler reaches 4° C., pause the program. Transfer the tubes/plate prepared in step 13 to the pre-chilled thermal cyclerand resume the cycling program.

TABLE 7
Cycling conditions for ligation

| | | |
|---|---|---|
| Step | Incubation temperature | Incubation time |

| | | | | |
|---|---|---|---|---|
| 1 | 4° | C. | 1 | min |
| 2 | 20° | C. | 15 | min |
| 3 | 65° | C. | 20 | min |

| | | | |
|---|---|---|---|
| 4 | 4° | C. | Hold |

Upon completion, place the reactions on ice and proceed with “Ligation cleanup reaction”. Alternatively, the samples can be stored at −20° C. in aconstant-temperature freezer for up to 3 days.

### Ligation Cleanup Reaction.

After ligation, transfer sample to ice, each sample add 2 μl of ligation cleanupreagent. Briefly centrifuge, mix by pipetting up and down 7-8 times and briefly centrifuge again. Program the thermal cycler according to Table 8. Use the instrument's heated lid. Before adding the tubes/plate to a thermal cycler, start the program. When thethermal cycler reaches 4° C., pause the program. Important: The thermal cycler must be pre-chilled and paused at 4° C. Transfer the tubes/plate prepared in step 18 to the pre-chilled thermal cycler and resume the cycling program.

TABLE 8
Cycling conditions for ligation cleanup reaction

| | | |
|---|---|---|
| Step | Incubation temperature | Incubation time |

| | | | | |
|---|---|---|---|---|
| 1 | 4° | C. | 1 | min |
| 2 | 37° | C. | 15 | min |
| 3 | 95° | C. | 10 | min |

| | | | |
|---|---|---|---|
| 4 | 4° | C. | Hold |

Upon completion, allow the thermal cycler to return to 4° C. Place the samples on ice and immediately proceed with “Target enrichment,” below.

### Target Enrichment TEPCR Reaction.

Prepare the target enrichment mix according to Table 9. Briefly centrifuge, mixby pipetting up and down 7-8 times and briefly centrifuge again.

TABLE 9
Reaction mix for target enrichment

| Component | Volume/reaction | |
|---|---|---|
| Adapter-ligated DNA from “Ligation | 30 | μl |
| cleanup reaction” (already in tube) | | |
| TEPCR buffer, 5x | 10 | μl |
| QIAseq Targeted DNA Pro Panel | 6.3 | μl |
| SmP-IL5 TEPCR-F Primer | 2 | μl |
| TEPCR modifier | 1.95 | μl |
| QN Taq Polymerase | 2.4 | μl |
| Total | 52.65 | μl |

Program a thermal cycler using the cycling conditions in Table 10 (panel with <12,000 primers/tube) or Table 11 (panel with ≥12,000 primers/tube).

TABLE 10
Cycling conditions for target enrichment
if number of primers <12,000/tube

| | | |
|---|---|---|
| Step | Time | Temperature |

| | | | |
|---|---|---|---|
| Initial denaturation | 2 | min | 98° C. |
| 8 cycles | 15 | s | 98° C. |
| | 2 | min | 65° C. |
| 1 cycle | 3 | min | 72° C. |

| | | |
|---|---|---|
| Hold | ∞ | 4° C. |

TABLE 11
Cycling conditions for target enrichment
if number of primers ≥12,000/tube

| | | |
|---|---|---|
| Step | Time | Temperature |

| | | | |
|---|---|---|---|
| Initial denaturation | 2 | min | 98° C. |
| 8 cycles | 15 | s | 98° C. |
| | 5 | min | 65° C. |
| 1 cycle | 3 | min | 72° C. |

| | | |
|---|---|---|
| Hold | ∞ | 4° C. |

Place the target enrichment reaction in the thermal cycler and start the run. After the reaction is complete, place the reactions on ice and proceed with “TEPCR cleanup reaction”, below. Alternatively, the samples can be stored at −20° C. in a constant-temperature freezer for up to 3 days.

### TEPCR Cleanup Reaction.

After TEPCR, transfer sample to ice, mix each TEPCR reaction by pipetting and transfer 20 μl from each sample to a new PCR tube. Note: The rest of TEPCR reaction can be stored at −20° C. if needed. Prepare the TEPCR cleanup mix according to Table 12. Briefly centrifuge, mix by pipetting up and down 7-8 times and briefly centrifuge again.

TABLE 12
Reaction mix for TEPCR cleanup

| Component | Volume/reaction |
|---|---|
| Target enriched DNA from | 20 μl |
| “Target enrichment TEPCR reaction” | |
| TEPCR cleanup reagent | 5 μl |
| Total | 25 μl |

Program the thermal cycler according to Table 13. Use the instrument's heated lid. Before adding the tubes/plate to a thermal cycler, start the program. When thethermal cycler reaches 4° C., pause the program. Important: The thermal cycler must be pre-chilled and paused at 4° C. Transfer the tubes/plate prepared in step 29 to the pre-chilled thermal cyclerand resume the cycling program.

TABLE 13
Cycling conditions for TEPCR cleanup reaction

| | | |
|---|---|---|
| Step | Incubation temperature | Incubation time |

| | | | | |
|---|---|---|---|---|
| 1 | 4° | C. | 1 | min |
| 2 | 37° | C. | 15 | min |
| 3 | 95° | C. | 10 | min |

| | | | |
|---|---|---|---|
| 4 | 4° | C. | Hold |

Upon completion, allow the thermal cycler to return to 4° C. Place the samples on ice and immediately proceed with “Universal PCR”, below.

### Universal PCR.

Prepare the Universal PCR in cleaned target-enriched DNA from TEPCR cleanup reaction according to Table 14. Briefly centrifuge, mix by pipetting up and down 7-8 times and briefly centrifuge again. For the QIAseq Targeted DNA Pro UDI plates, pierce the foil seal associated with each well that will be used, and transfer 5 μl (each well contains a forward primer and a reverse primer, each with a unique index) to the cleaned target-enriched DNA from “TEPCR cleanup reaction” sample tube/plate according to Table 14. Important: Only one UDI pair should be used per universal PCR reaction. Important: The QIAseq Targeted DNA Pro UDI index plates are stable for a maximum of 10 freeze-thaw cycles. If all 96-wells have not been used at one time, cover used wells with foil and return to the freezer. Do not reuse wells from the QIAseq Targeted DNA Pro UDI index plates once the foil seals have been pierced. This would risk significant cross-contamination.

TABLE 14
Reaction components for universal PCR if using
QIAseq Targeted DNA Pro UDI (12) or QIAseq Targeted
DNA Pro UDI Set A, B, C and D (96).

| Component | Volume/reaction | |
|---|---|---|
| Cleaned target-enriched DNA | 25 | μl |
| from “TEPCR cleanup reaction” | | |
| UPCR Buffer, 5x | 20 | μl |
| Index primers from QIAseq | 5 | μl |
| Targeted DNA Pro UDI index plate* | | |
| QN Taq Polymerase | 4.8 | μl |
| Nuclease-Free Water | 45.2 | μl |
| Total | 100 | μl |

*Applies to QIAseq Targeted DNA Pro UDI (12) or QIAseq Targeted DNA Pro UDI Set A, B, C, and D (96).

In a layout of InP-IL5-QUDI-## and InP-IL7-QUDI-##Index Primer Plate in QIAseq Targeted DNA Pro UDI (12) and QIAseq Targeted DNA Pro UDI Set A, B, C and D (96), each well contains one pair of pre-dispensed sample index primers plus universal primers for a single reaction, in universal PCR step.

Program a thermal cycler using the cycling conditions in Table 15 (cyclingprogram) and Table 16 (cycle number).

TABLE 15
Cycling conditions for Universal PCR

| | | |
|---|---|---|
| Step | Time | Temperature |

| | | | |
|---|---|---|---|
| Initial denaturation | 2 | min | 98° C. |
| Number of cycles (see Table 16) | 15 | sec | 98° C. |
| | 30 | sec | 62° C. |
| 1 cycle | 3 | min | 72° C. |

| | | |
|---|---|---|
| Hold | ∞ | 4° C. |

TABLE 16
Amplification cycles for Universal PCR

| Primers per pool | Cycle number |
|---|---|
| 6-24 | 33 |
| 25-96 | 31 |
| 97-288 | 29 |
| 289-1056 | 28 |
| 1057-3072 | 27 |
| 3073-5999 | 26 |
| 6000-12,000 | 25 |
| ≥12,000 | 24 |

After the reaction is complete, place the reactions on ice and proceed to “Cleanup of Universal PCR”, next step. Alternatively, the samples can be stored at −20° C. in a constant-temperature freezer for up to 3 days.

### Cleanup of Universal PCR.

Add 80 μl QIAseq Beads to finished universal PCR reaction, mix well by pipettingup and down several times. Incubate for 5 min at room temperature. Place the tubes/plate on magnetic rack for 5 min to separate beads from supernatant. Once the solution has cleared, with the tubes/plate still on the magnetic stand, carefully remove and discard the supernatant. Important: Do not discard the beads as they contain the DNA of interest. With the tubes/plate still on the magnetic stand, add 100 μl H2O to the bead, then add 80 μl QIAseq bead binding buffer. Take the tubes/plate off the magnetic stand, mix well by pipetting up and down several times. Return the tubes/plate to the magnetic rack for 5 min. Once the solution has cleared, with the tubes/plate still on the magnetic stand, carefully remove and discard the supernatant. Important: Do not discard the beads as they contain the DNA of interest. With the tubes/plate still on the magnetic stand, add 200 μl 80% ethanol to wash the beads. Carefully remove and discard the wash. Repeat the ethanol wash. Important: Completely remove all traces of the ethanol wash after this second wash. Remove the ethanol with a 200 μl pipet first, and then use a 10 μl pipet to remove any residual ethanol. With the tubes/plate still on the magnetic stand, air dry at room temperature for 10 min. Note: Visually inspect that the pellet is completely dry. Remove the tubes/plate from the magnetic stand and elute the DNA from the beads by adding 30 μl nuclease-free water. Mix well by pipetting. Return the tubes/plate to the magnetic rack until the solution has cleared. Transfer 28 μl supernatant to clean tubes or plate. The library can be stored in a −20° C. freezer prior to quantification using the QIAseq Library Quant System. Amplified libraries are stable for several months at −20° C. Once quantification is performed proceed to sequencing.

### Analyze the Library Using Agilent 2100 Bioanalyzer

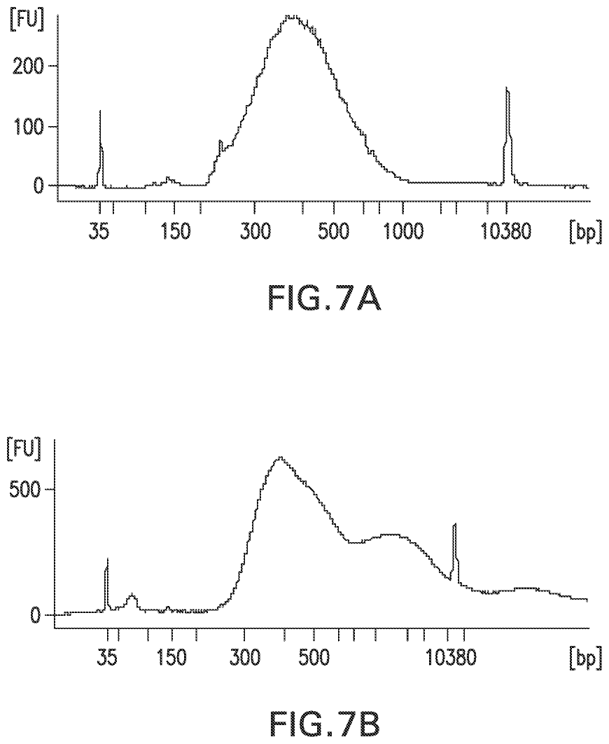

After the library is constructed and purified, the Bioanalyzer can be used to check the fragment size and concentration with the High Sensitivity DNA Kit. Libraries prepared for Illumina instruments demonstrate a size distribution between 200-1000 bp (FIG. 7A). Library over-amplification is normal (FIG. 7B) and this should not affect the sequencing results. Over-amplified libraries are usually single-stranded libraries with correct size but appear as “larger fragments” due to secondary structure. Amounts of DNA under the appropriate peaks can be used to quantify the libraries. However, due to the superior sensitivity and broad range of qPCR, we recommend quantifying the libraries using the QIAseq Library Quant System, especially when there are over-amplified libraries. FIG. 7A shows a library (without over-amplification) prepared for Illumina instruments. FIG. 7B shows a library (with over-amplification) prepared for Illumina instruments.

The foregoing description of the specific embodiments will so fully reveal the general nature of the invention that others can, by applying knowledge within the skill of the art, readily modify and/or adapt for various applications, without departing from the general concept of the invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be herein incorporated by reference.

What is claimed is:

1. A method of preparing a DNA library, comprising
(a) ligating an adapter to a target DNA molecule to generate a ligation product, wherein the adapter comprises one or more deoxyuridines and a unique molecular index (UMI);
(b) treating the ligation product with a uracil-DNA glycosylase (UDG); and
(c) amplifying the target DNA molecule attached to the adapter in the ligation product of (b) by single primer extension with a target specific primer to generate a target amplification product, wherein the target specific primer comprises one or more deoxyuridines and a target specific sequence.

2. The method of claim 1, wherein the target DNA molecule has been end-repaired.

3. The method of claim 1 or 2, wherein the target DNA molecule has been adenylated.

4. The method of any one of claims 1-3, wherein the target specific sequence comprises one or more deoxyuridines.

5. The method of any one of claims 1-4, wherein the target specific primer comprises a tag sequence comprising at least 2 deoxyuridines and a target specific sequence comprising 0-4 deoxyuridines in a 5′ to 3′ direction.

6. The method of any one of claims 1-5, further comprising treating the target amplification product with a UDG and then amplifying the target amplification product with a universal primer to generate a second target amplification product, wherein the universal primer does not comprise a deoxyuridine.

7. The method of claim 6, wherein the universal primer further comprises a sample index.

8. The method of any one of claims 1-7, wherein the ligating is performed at a pH of 8-9.

9. The method of claim 8, wherein the ligating is performed at a PEG concentration of 4% PEG to less than 10% PEG.

10. The method of any one of claims 1-9, wherein the amplifying is performed with an antibody based thermostable polymerase.

11. The method of any one of claims 6-10, further comprising purifying the second target amplification product with beads.

12. The method of claim 11, wherein the beads are solid phase reversible immobilization (SPRI) beads.

13. The method of any one of claims 1-12, wherein the DNA library can be used for next generation sequencing, profiling of DNA variants, human identity or paternity testing, pain or ADME pharmacogenomics, or detection of a genetic disease.

14. A method of preparing a DNA library, comprising amplifying a target DNA molecule with a single target specific primer to generate a target amplification product, wherein the target specific primer comprises a tag sequence, one or more deoxyuridines, and a target specific sequence.

15. The method of claim 14, wherein the adapter is a single adapter that is annealed to the target DNA molecule and extended, wherein the adapter comprises a UMI and a target specific sequence that does not contain a deoxyuridine.

16. The method of claim 14, wherein a pair of adapters are annealed to the target DNA molecule and extended, wherein each adapter of the pair of adapters comprises a UMI, a first or second target specific sequence, and one or more deoxyuridines.

17. The method of claim 14, wherein an adapter has been ligated to the target DNA molecule to generate a ligation product, wherein the adapter comprises one or more deoxyuridines and a unique molecular index (UMI).

18. The method of claim 17, wherein the target DNA molecule has been end-repaired.

19. The method of claim 17 or 18, wherein the target DNA molecule has been adenylated.

20. The method of any one of claims 17-19, wherein the ligating is performed at a pH of 8-9.

21. The method of any one of claims 17-20, wherein the ligating is performed at a PEG concentration of 4% PEG to less than 10% PEG.

22. The method of any one of claims 16-21, wherein the target specific primer comprises a tag sequence comprising at least 2 deoxyuridines and a target specific sequence comprising 0-4 deoxyuridines in a 5′ to 3′ direction.

23. The method of any one of claims 14-22, further comprising treating the target amplification product with a UDG and then amplifying the target amplification product with a universal primer to generate a second target amplification product, wherein the universal primer does not comprise a deoxyuridine.

24. The method of claim 23, wherein the universal primer comprises a sample index.

25. The method of any one of claims 14-24, wherein the amplifying is performed with an antibody based thermostable polymerase.

26. The method of any one of claims 21-25, further comprising purifying the second target amplification product with beads.

27. The method of claim 26, wherein the beads are solid phase reversible immobilization (SPRI) beads.

28. The method of any one of claims 14-27, wherein the DNA library can be used for next generation sequencing, profiling of DNA variants, human identity or paternity testing, pain or ADME pharmacogenomics, or detection of a genetic disease.

29. A DNA library made by the method of any one of claims 1-28.

30. A kit comprising a uracil-DNA glycosylase (UDG), an adapter comprising one or more deoxyuridines and a unique molecular index (UMI), a universal primer, and an antibody based thermostable polymerase.

31. The kit of claim 30, further comprising a ligase and a ligation buffer capable of providing a pH of 8 to 9 to a ligation reaction.

32. The kit of claim 31, wherein the ligation buffer is capable of providing a PEG concentration of 4% to less than 10% to the ligation reaction.

33. The kit of any one of claims 30-32, further comprising solid phase reversible immobilization (SPRI) beads.

34. The kit of any one of claims 30-33, further comprising a target specific primer comprising one or more deoxyuridines and a target specific sequence,


SEQUENCE LISTING (5 sequences)

SEQ ID NO: 1 - DNA, 59 bp, Artificial Sequence
  Adapter
  misc_feature (33)..(44): n is a, c, g, or t
cgucggcagcgucagatgtgtauaagagacagnnnnnnnnnnnnatgcautcgagucat

SEQ ID NO: 2 - DNA, 32 bp, Artificial Sequence
  I5-F forward primer
cgtcggcagcgtcagatgtgtataagagacag

SEQ ID NO: 3 - DNA, 21 bp, Artificial Sequence
  Target specific primer
aatgtacaguattgcgtttug

SEQ ID NO: 4 - DNA, 71 bp, Artificial Sequence
  I5IP index primer
aatgatacggcgaccaccgagatctacacatggccgacttcgtcggcagcgtcagatgtgtataagagaca

SEQ ID NO: 5 - DNA, 89 bp, Artificial Sequence
  I7IP index primer
caagcagaagacggcatacgagattgaacgttgtgtgactggagttcagacgtgtgctcttccgatctccagtacagtattgcgttttg